(19) United States
(12) Patent Application Publication
(10) Pub. No.: US 2007/0264373 A1
(43) Pub. Date: Nov. 15, 2007

(54) Toxin Induced Sympathectomy

(76) Inventors: Ian Carroll, Mountain View, CA (US); David Clark, Palo Alto, CA (US); Sean Mackey, Los Altos, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/587,535

(22) PCT Filed: Jan. 26, 2005
(86) PCT No.: PCT/US05/03081

§ 371 Date: Apr. 5, 2007

Related U.S. Application Data
(60) Provisional application No. 60/539,476, filed on Jan. 26, 2004.

(51) Int. Cl. A61K 35/74; A61P 23/00; A61P 43/00; A61P 9/00; A61P 9/10

(52) U.S. Cl. 424/780

(57) ABSTRACT

A selective and extended sympathetic nerve block is achieved by administration of a botulinum toxin at and/or around a targeted sympathetic ganglion. The toxin induced sympathetic block finds use for the treatment of sympathetically maintained pain. The toxin induced sympathetic block is also used to treat cardiovascular conditions of sympathetically maintained vasoconstriction; and of undesirable smooth muscle spasm.

The sympathetic nervous system is partially located to the sympathetic chain, which connects to skin, blood vessels and organs in the body cavity. The sympathetic chains are located on both sides of the spine. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion.

The synapses in the sympathetic ganglion use acetylcholine as a neurotransmitter; and the synapses of the post-ganglionic neurons use the neurotransmitters norepinephrine or acetylcholine. Acetylcholine activates at least two types of receptors, muscarinic and nicotinic receptors. The muscarinic receptors are found in effector cells stimulated by the postganglionic cholinergic neurons of the sympathetic nervous system. Nicotinic receptors are found in the synapses between the preganglionic and postganglionic neurons of both the sympathetic and parasympathetic nervous system.

Among the effects of sympathetic nerve stimulation are increased heart rate, reduced intestinal motility, dilation of the pupils, reduced salivation, and increased conversion of glycogen to glucose in the liver.

Sympathetic nerve blocks are used to interrupt sympathetic nerve transmission. Typically, such blocks are performed with a needle placed percutaneously at the stellate ganglia, lumbar sympathetic ganglia, celiac plexus, superior hypogastric plexus, inferior hypogastric plexus, thoracic sympathetic chain, pterygopalantine ganglia, or ganglia impar. Once the needle is placed, a local anesthetic is administered, which blocks the sympathetic and other nerves passing through the afore-mentioned ganglia. In conventional methods of sympathetic block, the drug usually injected is the local anesthetic bupivacaine. Epinephrine or clonidine is often included to prolong the effect. Other drugs may include corticosteroids to reduce inflammation, and clonidine to enhance the anesthetic effect.

The effects of a sympathetic nerve block vary, depending on the site of the block, and may include increased regional blood flow, oxygen delivery, and decrease of sympathetically maintained pain. Sympathetic blocks can also alter the function of organs that have a sympathetic control component.

Pain is the most common symptom for which patients seek medical assistance and relief, and chronic pain is among the most vexing problems that physicians face. Pain lasting over six months in duration or pain persisting beyond the time of expected tissue healing is typically considered chronic. Chronic pain can be unresponsive to analgesic agents, making its relief particularly difficult.

In some cases, pain conditions are maintained after the underlying cause has healed. In at least some of such patients, there is a sympathetic maintenance of pain. In order to diagnose sympathetically maintained pain, a sympathetic block may be performed. The most common approach is to perform a local anesthetic sympathetic trunk block: a lumbar paravertebral sympathetic block for the lower extremity and a stellate ganglion block or upper thoracic sympathetic block for upper extremity symptoms. However all of the above mentioned sympathetic ganglia have been blocked with local anesthetic for therapeautic and diagnostic purposes. Other diagnostic tests include administration of an oral adrenergic antagonist, such as phentolamine intravenously (IV).

For example, complex regional pain syndrome (CRPS) is a regional, post-traumatic, neuropathic pain problem that most often affects one or more limbs. Most patients with CRPS have an identifiable inciting or initiating injury, which may be trivial or severe. The key features are pain, allodynia and hyperalgesia, abnormal vasomotor activity, and abnormal sudomotor activity persisting beyond the period of normal healing. Pain usually spreads beyond the area of the initial injury and, in its most severe form, may involve the entire limb and, rarely, the contralateral limb. Regional anesthesia techniques, including sympathetic blocks have been used for treatment.

In addition to short-term anesthetics and analgesics, neurolytic techniques have also been used as a sympathetic block. Nerve destruction can be accomplished by the injection of ethanol, phenol, or other neurolytic agents, usually at sites where a test injection of local anesthetic has produced pain relief or other desired sympatholytic effect. Repetitive anesthetic blocks can also eventually destroy the ganglion, causing a virtual sympathectomy. Alternative procedures include open surgical sympathectomy, or radiofrequency ablation of the sympathetic nerve (more generally). However, surgical sympathectomy may result in a compensatory hyperhidrosis: a condition characterized by abnormally profuse sweating in a location remote form the sympathectomy. Other complications can also occur, including paresis, paralysis, and bowel or bladder dysfunction. Neurolytic procedures typically can only be considered if all other measures have failed.

The bacterium Clostridium botulinum produces the polypeptide neurotoxin, botulinum toxin. Seven immunologically distinct botulinum neurotoxins have been characterized: botulinum neurotoxin serotypes A, B, C₁, D, E, F and G, each of which is distinguished by neutralization with type-specific antibodies. The different serotypes of botulinum toxin vary in the animal species that they affect and in the severity and duration of the paralysis they evoke. Botulinum toxin apparently binds with high affinity to cholinergic motor neurons, is translocated into the neuron and blocks the release of acetylcholine.

Although all the botulinum toxins apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes.

The toxins are released by the bacterium as complexes comprising a 150 kD botulinum toxin protein molecule, along with associated non-toxin proteins. The botulinum toxin type A complex is produced as 900 kD, 500 kD and 300 kD forms. Botulinum toxin types B and C₁ are produced as a 500 kD complex; toxin type D is produced as both 300 kD and 500 kD complexes; and toxin types E and F are produced as 300 kD complexes. The complexes contain a non-toxin hemaglutinin protein and nonhemaglutinin protein. These two non-toxin proteins may act to provide stability against denaturation to the botulinum toxin molecule and protection against digestive acids when toxin is ingested.

All the botulinum toxin serotypes are initially synthesized as inactive single chain proteins, which are cleaved or nicked by proteases to become neuroactive. The bacterial strains that make botulinum toxin serotypes A and G possess endogenous proteases, while the other serotypes may need to be activated.

Botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine, CGRP and glutamate.

A commercially available botulinum toxin containing pharmaceutical composition is sold under the trademark BOTOX™, which consists of a purified botulinum toxin type A complex, albumin and sodium chloride packaged in sterile, vacuum-dried form. BOTOX™ can be reconstituted with sterile, non-preserved saline prior to intramuscular injection. MYOBLOC, Botulinum toxin type B is also commercially available, and other serotypes are under active development by a variety of pharmaceutical firms.

The FDA has approved the use of BOTOX™ for cervical dystonia, as well as strabismus, and blepharospasm. It has also been used widely for cosmetic purposes and widely injected into muscles for myofascial pain and migraine prophylaxis. For cervical dystonia, the administered dose is usually about 198 to 300 units. A typical dose when used for myofascial pain or headache is 100 units. Toxicity is uncommon and most often relates to weakness of the muscle that has been injected, or spread of weakness to adjacent muscles that have been inadvertently injected. Therefore, when used to treat cervical dystonia the most frequently reported adverse reaction were dysphagia, upper respiratory infection, neck pain, and headache. Other events reported in a small number of patients included: increased cough, flu syndrome, back pain, rhinitis, dizziness, hypertonia, soreness at injection site, asthenia, oral dryness, speech disorder, fever, nausea and drowsiness. When used for strabismus, weakness of adjacent muscles may lead to ptosis and vertical deviation.

Current methods of sympathetic block suffer from several deficiencies. When a local anesthetic is used, the block acts for only a short time. In some cases, only a partial block may be achieved. When a neurolytic agent is used, axonal damage results leading to unpredictable regrowth of the nerves. Additionally, significant scarring in the area occurs, which limits the ability to perform repeated neurolytic blocks. Finally, spread of the neurolytic solution can occur to other structures leading to significant neurological or vascular injury. There are a number of conditions that would benefit from a more complete, and longer acting sympathetic block, which does not permanently damage the nerve. Among these conditions is chronic pain, for which a substantial number of persons can find no meaningful relief. Therapeutic methods that can address the issues are of great clinical interest.

Publications

Reviews of some of the medical uses of botulinum toxin are described by Walker (2003) Phys Med Rehabil Clin N Am. 14(4):749-66; Delgado (2003) J Am Acad Orthop Surg. 11(5):291-4; Jankovic (2004) Adv Neurol. 94:275-86; Harrison (2003) Curr Opin Opthalmol. 14(5):241-5; and Pahwa and Lyons (2003) Am J Med. 115(2):134-42. The use of botulinum toxin is also discussed, inter alia, by Donovan, U.S. Pat. No. 6,635,247, issued Oct. 21, 2003.

### SUMMARY OF THE INVENTION

Methods are provided for extended sympathetic nerve block, by administration of a neurotoxin at and/or around a targeted sympathetic ganglion. A preferred neurotoxin is a botulinum toxin, e.g. serotypes A, B, C₁, D, E, F and G. The methods provide for reliable, and reversible interruption of sympathetic nerve transmission, for prolonged periods of time. The block will generally last for longer than one week, and may last for longer than one month, and up to several months duration. The methods also provide for enhanced selectivity, and will not block visceral afferent nerve fibers. The neurotoxin may be administered percutaneously as one or a cocktail of serotypes; and is optionally combined with a local anesthetic, anti-inflammatory agent, and the like.

In one embodiment of the invention, the toxin induced sympathetic block is used for the treatment of sympathetically maintained pain. In this method, the involvement of sympathetic nerves may be initially diagnosed through a short term sympathetic block, for example by administration of an IV adrenergic antagonist; percutaneous administration of local anesthetic near the sympathetic ganglion or chain, and the like. Following confirmation of sympathetic involvement in pain, the neurotoxin is administered to the targeted sympathetic ganglion.

In one embodiment, a block of the upper extremities is established by administration of neurotoxin to the stellate ganglion, i.e. affecting the inferior, middle and superior cervical sympathetic nerves. In another embodiment, a block of the lower extremities is accomplished by administration of neurotoxin to the lumbar sympathetic ganglion.

In another embodiment of the invention, the toxin induced sympathetic block is used to treat cardiovascular conditions that are improved by an interruption of sympathetic stimulation, e.g. hypertentsion, and other conditions where sympathetically induced vasoconstriction adversely affects a patient's health. In yet another embodiment, the toxin is induced sympathetic block is used to treat conditions characterized by smooth muscle spasms, some of which smooth muscles may be arterial, e.g. in the treatment of cerebral vasospasm, coronary vasospasm, peripheral vasospasm and the like.

### DETAILED DESCRIPTION OF THE EMBODIMENTS

As used herein, the following terms have the meanings ascribed to them unless specified otherwise.

The singular forms “a,” “an,” and “the” include plural references unless the context clearly dictates otherwise. Unless defined otherwise, all technical and scientific terms used herein have the same meaning as commonly understood to one of ordinary skill in the art to which the present invention belongs. Although any methods, devices, and materials similar or equivalent to those described herein can be used in the practice or testing of the invention, the preferred methods, devices, and materials are described herein.

The term “neurotoxin” and “botulinum toxin” as used herein refer to polypeptide toxins that specifically interfere with the release of acetylcholine, e.g. across a synapse. Such toxins are known in the art, and have been isolated from various species of Clostridia, including Clostridium botulinum, Clostridium butyricum, Clostridium beratti and Clostridium tetani. For use in the subject methods, any of the native toxin forms, modifications thereof, or a combination of forms may be used.

The sequence of the toxin peptides may be altered from the native sequence in various ways known in the art to generate targeted changes. The altered peptide will usually be substantially similar to the sequences provided herein, i.e. will differ by one amino acid, and may differ by two, three or more amino acids. The sequence changes may be substitutions, insertions or deletions.

The toxin polypeptides may be joined to a wide variety of other oligopeptides or proteins for a variety of purposes. Various post-translational modifications may be achieved. Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids.

The toxin polypeptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

The neurotoxin is preferably a botulinum toxin, such as one of the botulinum toxin serotypes A, B, C₁, D, E, F or G. Preferably, the neurotoxin is botulinum toxin type A. In a preferred embodiment, the botulinum toxin is administered as a complex, in order to enhance the stability of the toxin.

Botulinum toxins for use according to the present invention may be provided in liquid form, or can be stored in lyophilized or vacuum dried form in containers under vacuum pressure. Prior to lyophilization the botulinum toxin can be combined with pharmaceutically acceptable excipients, stabilizers and/or carriers, such as albumin. The lyophilized or vacuum dried material can be reconstituted with saline or water, or local anesthetic or other medicine that do not degrade the Botulinum toxin, and may produce an independent neuromodulatory effect.

The dose of a neurotoxin administered according to the present invention can vary widely according to various patient variables including size, weight, age, disease severity, responsiveness to therapy, and solubility and diffusion characteristics of the neurotoxin toxin chosen. Methods for determining the appropriate route of administration and dosage are generally determined on a case by case basis by the attending physician. Such determinations are routine to one of ordinary skill in the art.

The pharmaceutical formulation comprising neurotoxin is administered in a dose that is effective for the desired effect; e.g. to relieve pain the desired dose will provide relief from pain for a sustained period of time.

Most preferably, the neurotoxin is administered in an amount of at least about 1 U/kg, usually at least about 2.5 U/kg; and not more than about 10 U/kg, usually not more than about 7.5 U/kg, and may be about 5 U/kg. For the purpose of sympathectomy, botulinum toxin A will be administered at the dose range of from about 0.01 to 10 U/kg, usually about 0.1 to about 5 U/kg; more usually about 0.1 to about 1 U/kg, or about 0.2 to about 0.5 U/kg; more usually in the vicinity of a sympathetic ganglion. This is expected to provide prolonged pain relief. The technique is similar for patients of all ages.

The term “unit dosage forms” refers to physically discrete units suitable as unitary dosages for a human subject. Each unit contains a predetermined quantity of active material calculated to produce the desired onset, tolerability, and therapeutic effects, in association with a suitable pharmaceutical excipient (e.g., an ampoule).

The term “administering” in the context of the present invention will refer to percutaneous administration at one or more of the ganglia in the sympathetic chain, which may be performed under fluoroscopy, direct vision, direct surgical vision, endoscopic delivery, and the like. These sites may include: superior cervical ganglia; middle superior cervical ganglion; vertebral ganglion; cervicothoracic (stellate) ganglion; sympathetic trunk; thoracic sympathetic ganglion; aorticorenal ganglion; celiac ganglion; superior mesenteric ganglion; inferior mesenteric ganglion; superior and inferior hypogastric plexus. In this manner, an indirect of nerves of interest is achieved, including, for example, the greater, lesser and least splanchnic nerves; lumbar splanchnic nerves; sacral splanchnic nerves; and the like.

The term “analgesia” refers generally to the reduction of pain or to the full elimination of pain in a human suffering from pain. As used herein, the term “analgesically effective amount” refers to an amount of a toxin or a combination of toxin and other agents that produces a reduction of sympathetically maintained pain or a full elimination of sympathetically maintained pain in a human patient. There are no animal models for sympathetically maintained pain. Preferably, the analgesically effective amount produces minimal toxic side-effects.

The terms “sustained activity,” “extended period of action,” and “long duration of effect” are used interchangeably herein to refer to the situation where a sympathetic block from a single dose, for example, when delivered by percutaneous injection, is maintained for at least about 1 week, preferably at least about 1 month, more preferably at least 2 months, or more.

The term “local anesthesia” refers to anesthesia characterized by the loss of sensation only in the area of the body where an anesthetic agent is administered. Local anesthesia can result, for example, from inhibition of nerve transmission at a nerve or nerves proximal to the site at which the painful stimulus is present. As used herein, the term “anesthetically effective amount” refers to an amount of an anesthetic agent or a combination of anesthetic agents that produces an anesthetic effect, e.g., a partial or total loss of sensation, inhibition of sensory perception, or inhibition of motor function.

The term “anesthetic” refers to an agent that causes loss of sensation in a human or other mammal with or without the loss of consciousness. More particularly, the term “local anesthetic” refers to an anesthetic agent that induces local anesthesia by reversibly inhibiting peripheral nerve excitation and/or conduction.

In some embodiments of the invention, e.g. in the treatment of sympathetically maintained pain, a local anesthetic is optionally combined with the neurotoxin during administration. The short-term anesthetics also find use in the diagnosis of sympathetic involvement in pain.

Anesthetics of interest for the methods of the invention include bupivacaine; levobupivacaine; lidocaine; prilocalne; mepivacaine; ropivocaine; cocaine; procaine; benzocaine; tetracaine; chloroprocaine; etidocaine; etc. These agents are used at conventional doses, as known in the art, e.g. in a preferred embodiment, bupivacaine at a concentration of from about 0.2% to 0.5% is administered in a single dose of 10 cc.

The term “corticosteroid” refers to any of the steroid hormones produced by the adrenal cortex or their synthetic equivalents, such as cortisol and aldosterone. Corticosteroids that are useful in the present invention to prolong in vivo nerve blockade include, but are not limited to, glucocorticoids such as dexamethasone, cortisone, hydrocortisone, prednisone, other glucocorticoids routinely administered orally or by injection, and pharmaceutically acceptable salts and combinations thereof. Other glucocorticoids include beclomethasone, betamethasone, flunisolide, methyl prednisone, paramethasone, prednisolone, triamcinolome, alclometasone, amcinonide, clobetasol, fludrocortisone, diflurosone diacetate, fluocinolone acetonide, fluoromethalone, flurandrenolide, halcinonide, medrysone, mometasone, and pharmaceutically acceptable salts and combinations thereof.

The term “buffering agent” refers to a substance that minimizes change in the acidity of a solution when an acid or base is added to the solution. Such buffering agents are well known to those skilled in the art. Typically, the compositions of the present invention are buffered, e.g., with bicarbonate, to maintain a mildly acidic or mildly alkaline pH. Buffering the solution also hastens the speed of onset and enhances the duration of drug effect. Generally, the compositions of the present invention are buffered to maintain the highest stability of the toxin during administration.

Permeability enhancers may also be included. Permeability enhancers are used to aid in the passage of an agent into a tissue or across a cell membrane. Typical enhancers may include bile salts such as sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate. Other permeation enhancers such as urea, sodium dodecyl sulfate (SDS), dimethyl sulfoxide (DMSO), sodium lauryl sulfate, salts and other derivatives of saturated and unsaturated fatty acids, surfactants, bile salt analogs, derivatives of bile salts, or such synthetic permeation enhancers as described in U.S. Pat. No. 4,746,508 may also be used.

Lipophilic and/or amphiphilic solvents can be added to the carrier to prolong nerve blockade. These materials are well known to those skilled in the art and available from a variety of commercial sources. Examples of solvents include alcohols such as ethanol added in a dosage equivalent to approximately 1% alcohol, polyoxyethylene sorbitan derivatives such as polysorbate-80 or Tween, added in a concentration equivalent to between 1% and 3%.

The toxin may be administered as a pharmaceutical formulation of one or a combination of toxins; e.g. botulinum toxin serotype A alone, or in combination with any one of botulinum toxin serotypes B, C₁, D, E, F and G. In a first embodiment, the pharmaceutical formulation further comprises a buffering agent. In a second embodiment, the pharmaceutical formulation further comprises a corticosteroid. Preferably, the corticosteroid is a glucocorticoid selected from the group consisting of dexamethasone, cortisone, hydrocortisone, prednisone, other glucocorticoids routinely administered orally or by injection, and pharmaceutically acceptable salts and combinations thereof. In a fourth embodiment, the pharmaceutical formulation further comprises a local anesthetic, as described above. In a fifth embodiment, the formulation is supplied as a liquid (e.g., solution). Preferably, the liquid contains the active agents and adjuvants in suitable concentrations for immediate use without further mixing or dilution. In a sixth embodiment, the local formulation is supplied as a lyophilized powder that can be reconstituted with water prior to injection.

The following guidelines are not intended to imply any limitation to the dose that may be used in the methods and compositions of the present invention, but may indicate some guidelines for formulation and methods of use.

### Sympathetically Maintained Pain

Many patients have refractory pain that is sympathetically maintained, particularly in the lower extremities, although other regions may also be involved. These patients experience severe pain that cannot be adequately controlled with medications, but is relieved by interruption of the sympathetic nervous system signals, by injecting local anesthetic at the site of the sympathetic ganglia.

Lumbar sympathetic blocks are part of the standard of care for treating patients with sympathetically-maintained pain (e.g. in complex regional pain syndrome or reflex sympathetic dystrophy—RSD). In these patients lower extremity pain can be reduced or abolished temporarily by blocking sympathetic nerves with a lumbar sympathetic block. However, the short time nature of the relief can be unacceptable, and patients who respond only transiently to sympathetic blocks often choose between potentially dangerous lumbar sympathetic block with neurolytic agents, surgical sympathectomy, continued severe refractory debilitating pain or other risky invasive surgical procedures such as spinal cord electrical stimulation.

The methods of the invention provide a selective and sustained relief from sympathetically maintained pain, by administration of a toxin, such as a botulinum toxin, at a targeted sympathetic ganglion. While any of the previously listed sites for administration may be used, certain sites will have particular utility. For the relief of pain in the lower extremities, a sympathetic lumbar block of the lumbar sympathetic ganglion will be used. For relief of pain in the upper extremities, a sympathetic clock at the stellate ganglion may be used.

Other targeted sites include the superior cervical ganglia for treatment of pain in the face and head, migraines; the sympathetic trunk for treatment of angina pain; a celiac plexus block for treatment of chronic abdominal pain; and the superior hypogastric plexus for treatment of pain from endometriosis and other pelvic pain.

In one embodiment of the invention, a patient suffering from chronic pain is diagnosed as having sympathetically maintained pain by the method of performing a diagnostic sympathetic block of the appropriate ganglion with a local anesthetic, e.g. with bupivacaine. Patients that have greater than about 50% documented pain relief for more than about 5 hours but less than about two weeks after a diagnostic sympathetic block are identified as having sympathetically-maintained pain.

Treatment with an analgesically effective dose of toxin is performed by injection of the dose in the retroperitoneal space at the border of the ganglion or vertebral body. The injection may be performed with a single or divided dose. Fluoroscopic guidance may be used to aid in the procedure.

Upon completion of the injection, the patient will have a selective block of acetylcholine release from the targeted sympathetic nerves, thereby preventing the maintenance of pain. The block provides pain relief for extended periods of time, but does not permanently damage the nerves. As the block wears off, the patient may have repeated blocks administered in order to maintain an acceptable quality of life.

### Cardiovascular and Smooth Muscle Conditions

The methods of toxin mediated sympathetic block, as set forth herein, also find use in the treatment of cardiovascular conditions that benefit from decreased sympathetic activation. Sympathetic activation causes vasoconstriction in regions such as the renal and splanchnic circulations, which redistributes cardiac output to the exercising muscles. Interruption of sympathetic stimulation of the cardiovascular system can provide for increased blood flow to targeted tissues.

Establishing a sympathetic block of the superior cervical ganglia is used to treat retinal artery thrombosis. This condition may be related to diabetes; which is also associated with diabetic foot injuries, which may be treated with a sympathetic lumbar block. Peripheral vascular disease may be treated with a block of the sympathetic trunk.

Other cardiovascular conditions can be related to atherosclerosis, e.g. coronary artery disease, which may be aided with a block of the sympathetic trunk; and post prandial ischemia, which may be aided by a celiac plexus block. Additional cardiovascular conditions include essential hypertension, which may be aided with a block of the sympathetic ganglion in the celiac plexus, aorticorenal ganglion; lumbar sympathetic ganglion; and celiac ganglion. Renal hypertension may be aided with a block of the sympathetic ganglion in the celiac plexus, aorticorenal ganglion; lumbar sympathetic ganglion; and celiac ganglion. The methods find use in preventing cerebral vasospasm, and/or may be used following subarrachnoid hemorrhage, cerebral vasospasm, and the like.

Vasospatic disease conditions that may be treated with the methods of the invention include Raynaud's Disease and Phenomenon. Raynaud's disease is idiopathic, while Raynaud's phenomenon is secondary to other conditions, such as connective tissue disorders, obstructive arterial diseases, neurogenic lesions, drug intoxications, dysproteinemias, myxedema, primary pulmonary hypertension, and trauma. In Raynaud's disease, the threshold for the vasospastic response is lowered by local cold or anything that activates sympathetic outflow or releases catecholamines. Raynaud's disease is differentiated from Raynaud's phenomenon by bilateral involvement and no evidence of an underlying cause. Previous methods of treatment have included regional sympathectomy.

Raynaud's phenomenon is also found with systemic sclerosis, which may cause rapidly progressive and often fatal visceral involvement. A cutaneous scleroderma may be referred to as CREST syndrome (Calcinosis, Raynaud's phenomenon, Esophageal dysfunction, Sclerodactyly, Telangiectasia). The most common initial complaints in systemic sclerosis are Raynaud's phenomenon and insidious swelling of the acral portions of the extremities, with gradual thickening of the skin of the fingers.

Conditions characterized by smooth muscle spasms include cerebral vasospasm, which is treated with a superior cervical ganglion block; coronary vasospasm, which is treated with a block of the thoracic sympathetic ganglion; vasospasm of the lower extremities, which is treated with a lumbar sympathetic ganglion block; and rectal spasm, which is treated with a block of the superior and inferior hypogastric plexus. The latter may also be used for the treatment of interstitial cystitis.

In other aspects of the invention, hyperhydrosis may be treated, e.g. by a sympathetic block of the superior cervical ganglion; or a block of the sympathetic trunk.

A block of the celiac plexus; including an indirect block of the greater, lesser and least splanchnic nerves; and/or lumbar splanchnic nerves; is useful in alleviating ischemic bowel, cirrhosis, pancreatitis, irritable bowel disease. This block may also find use in the treatment of interstitial cystitis.

In other aspects of the invention sympathetic block with Botulinum toxin may be used to treat dysfunctional labor and premature labor in obstetric patients.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to biological functional equivalency considerations, changes can be made in protein structure without affecting the biological action in kind or amount. All such modifications are intended to be included within the scope of the appended claims.

### Example 1

An 80 year old male reported with refractory RSD who had received prior local anesthetic lumbar sympathetic blocks with less than 5 days pain relief. The patient was administered a single injection of 0.1 U/kg botulinum A. No complications were reported, and the patient had significant relief of pain relief in excess of 6 months.

### Example 2

Patients with chronic severe refractory pain of the lower extremity that have had greater than 50% documented pain relief for more than 5 hours but less than two weeks after a standard lumbar sympathetic block are identified as having sympathetically-maintained pain, and are treated with the methods of the invention. Such patients will by definition have already been deemed appropriate for lumbar sympathetic block and will have undergone such block as a routine part of care.

Subjects are given a form asking them to rate their pain (from 0 to 10 where 0 is no pain and 10 is worst pain imaginable) at noon every day starting one week before the injection and continuing until they feel there pain has returned to baseline or two weeks whichever is longer. Days of analgesia is the primary endpoint of the study. In addition the patient is asked to fill out a form for a validated measurement of global functioning prior to the first block, and 4 weeks after each block. They are also asked to fill out a neuropathic pain scale (NPS) form for a validated measure of neuropathic pain each week of the study. In addition, 4 weeks after each block the patient undergoes a routine physical examination with testing for weakness and quantitative sensory change. During this examination and prior to treatment the treated leg is assessed for sweating and temperature.

Treatment

Lumbar sympathetic block are performed twice on each patient: once as in the standard of practice with 10 cc 0.5% Bupivacaine; and once with 75 units of Botox in 10 cc of 0.5% Bupivacaine. These are done in random order and the investigator and the patient are blinded to which medicine they received. Two weeks after their pain has returned to 75% of their baseline the patient receives the second of the blocks.

Lumbar sympathetic blockade is accomplished by placing an IV in the patient. The patient is then placed face down on a fluoroscopy table; prepped and draped in a sterile fashion. Conscious sedation is provided with versed and Fentanyl in the operating room with monitoring of blood pressure, pulse oximetry, and EKG. The skin is topicalized with one cc of 1% Lidocaine.

The L2 lumbar vertebral body is identified and under fluoroscopic guidance a 22 gauge 6 inch spinal needle is placed at the anterolateral border of the L2 vertebral body. The retroperitoneal space is identified with a loss of resistance technique. Correct needle positioned is confirmed radiographically and appropriate spread of medications is verified by injecting 3 cc of radio-opaque contrast material. The needle is aspirated to verify that it is not intravascular. Ten cc of Bupivacaine 0.5% is injected in divided dose to ensure safety. Between injections the patient is asked to report ringing in the ears or tingling in the mouth.

### Example 3

An 63 year old man with ischemic right foot who received improvement in blood flow from an lumbar epidural placement with local anesthetic, but whose pain and ischemic symptoms returned after removal of the catheter. A lumbar sympathetic block with 75 units of Botox in 10 cc of 0.5% bupivacaine was performed with marked improvement in pain and blood flow to the extremity allowing discharge from the hospital. This has lasted in excess of 3 months.

### Example 4

A total of 6 patients with peripheral vascular disease have undergone lumbar sympathetic block as described in Example 2. Patients were treated with 100 units botulinum type A. Four of the patients experienced greater than 50% pain relief while ambulating, an effect which lasted for at least 2 months.”

### Example 5

A 50 year old with refractory pain in the foot received improvement with lumbar sympathetic blocks performed with local anesthetics. Relief was partial and only lasted several days in a stereotypic manner. She received 75 units of Botox in 10 cc of 0.5% bupivacaine in a Lumbar sympathetic block. She achieved more profound pain relief as assessed by visual analog scales and health related quality of life measures. This pain relief lasted more than 6 months.

### Example 6

A 52 year old woman with pain in her foot had only temporary relief with lumbar sympathetic blocks with bupivacaine alone. Following repetition of the block with Botox, 75 units placed at the lubar sympathetic ganglia, she reported more profound pain relief lasting more than two months.

What is claimed is:

1. A method for treating sympathetically maintained chronic pain, the method comprising:
administering a therapeutically effective dose of a botulinum toxin type A, B, C₁, D, E, F or G to a sympathetic ganglion of a human patient, thereby achieving a sympathetic block for an extended period of time.

2. The method according to claim 1, wherein said botulinum toxin is botulinum toxin type A.

3. The method according to claim 2, wherein said effective dose of botulinum toxin is from about 1 to 300 units.

4. The method according to claim 3, wherein said sympathetically maintained chronic pain is of the lower extremities, and said block is of the lumbar splanchnic nerves.

5. The method according to claim 3, wherein said sympathetically maintained chronic pain is of the upper extremities, and said block is of the inferior, middle or superior cervical sympathetic ganglion.

6. The method according to claim 3, wherein said sympathetic ganglion is one or more of the superior cervical ganglia; middle superior cervical ganglion; vertebral ganglion; cervicothoracic (stellate) ganglion; sympathetic trunk; thoracic sympathetic ganglion; aorticorenal ganglion; lumbar sympathetic ganglion; celiac ganglion; superior mesenteric ganglion; inferior mesenteric ganglion; superior and inferior hypogastric plexus; and ganglion impar.

7. The method according to claim 3, wherein said method further comprises the steps of:
identifying the chronic pain as being mediated by the sympathetic nervous system by administering a local anesthetic as a sympathetic block;
wherein a cessation of at least about 50% of the perceived pain for a short period of time following said sympathetic block is indicative of sympathetically maintained pain.

8. A method for treating cardiovascular conditions, the method comprising:
administering a therapeutically effective dose of a botulinum toxin type A, B, C₁, D, E, F or G to a sympathetic ganglion of a human patient, thereby achieving a sympathetic block for an extended period of time.

9. The method according to claim 8, wherein said cardiovascular condition is selected from the group consisting of retinal artery thrombosis; peripheral vascular disease; coronary artery disease; post prandial ischemia; cerebral vasospasm; coronary vasospasm; Raynaud's Disease, Raynaud's Phenomenon, and vasospasm of the lower extremities.

10. The method according to claim 9, wherein said treatment provides for pain relief in said patient.

11. The method according to claim 9, wherein said botulinum toxin is botulinum toxin type A.

12. The method according to claim 11, wherein said effective dose of botulinum toxin is from about 1 to 300 units.

13. A method of treating a disease with a sympathetic block of the celiac plexus, the method comprising:
administering a therapeutically effective dose of a botulinum toxin type A, B, C₁, D, E, F or G to the celiac plexus of a human patient, thereby achieving a sympathetic block for an extended period of time.

14. The method according to claim 13, wherein said condition is selected from the group consisting of:
ischemic bowel, cirrhosis, pancreatitis, irritable bowel disease, and interstitial cystitis.

15. The method according to claim 13, wherein said botulinum toxin is botulinum toxin type A.

16. The method according to claim 15, wherein said effective dose of botulinum toxin is from about 1 to 300 units.
